(19) United States
(12) Patent Application Publication
(10) Pub. No.: US 2011/0039768 A1
(43) Pub. Date: Feb. 17, 2011

(54) FISH PROTEIN HYDROLYSATE HAVING A SATIETOGENIC ACTIVITY, NUTRACEUTICAL AND PHARMACOLOGICAL COMPOSITIONS COMPRISING SUCH A HYDROLYSATE AND METHOD FOR OBTAINING SAME,

(76) Inventors: Hubert Drieu La Rochelle, Saint-Malo (FR); Elisa Courois, Saint-Malo (FR); Benoit Cudennec, Quimper (FR); Martine Fouchereau-Peron, Tregunc (FR); Rozenn Ravallec-Ple, Loos (FR)

Correspondence Address: THORPE NORTH & WESTERN, LLP., P.O. Box 1219, SANDY, UT, 84091-1219, US

(21) Appl. No.: 12/866,878

(22) PCT Filed: Feb. 12, 2009
(86) PCT No.: PCT/EP09/51638

§ 371 Date: Oct. 6, 2010

(30) Foreign Application Priority Data
Feb. 12, 2008 (FR) 08/00751

(51) Int. Cl. A61K 38/02; A61P 3/10; A61P 3/06; A61P 3/04; A61P 9/12; A61P 9/10; C07K 1/00

(52) U.S. Cl. 514/4.9; 514/7.2; 514/6.9; 514/7.4; 514/1.1; 514/12.6; 435/272

(57) ABSTRACT

The present invention relates to a fish protein hydrolysate containing molecules capable of exerting a satietogenic activity and of regulating food intake in humans or animals. More specifically, the protein hydrolysate according to the invention enables stimulation of the secretion of endogenous cholescystokinins (CCKs) and of endogenous glucagon-like peptide 1 (GLP1) molecules by intestinal cells and the supply of exogenous CCKs. The fish protein hydrolysate according to the invention is obtained by enzymatic hydrolysis of at least one protein source selected from the group composed of the pelagic fish species Micromesistius poutassou, Clupea harengus, Scomber scombrus, Sardina pilchardus, Trisopterus esmarki and Trachurus spp., the demersal fish species Gadus morhua, Pollachius virens, Melanogrammus aeglefinus and Coryphaenoides rupestris, and the species of fish belonging to the order Siluriformes, said enzymatic hydrolysis being carried out by means of a mixture of enzymes comprising endopeptidases derived from Bacillus amyloliquefaciens and from Bacillus licheniformis, or derived from Bacillus amyloliquefaciens, from Bacillus licheniformis and from Aspergillus oryzae.

The present invention concerns a fish protein hydrolysate containing molecules immunologically related to the gastrin/cholecystokinin family and able to exert a satietogenic activity and regulate food intake in humans or animals. The invention also concerns a method of obtaining such a fish protein hydrolysate as well as a composition, a food product, a food supplement or a medication comprising such a fish protein hydrolysate.

Obesity is being observed more and more within the population and is becoming a constant preoccupation. Such a phenomenon is the result of imbalance between the mean energy intake and the total energy expenditure. This is because, when the organism receives more than it expends, it stores some of the addition of energy in the form of fat in the adipocytes making up the adipose tissue. These cells swell and then cause a visible weight gain. They may then arrive at saturation and multiply. Obesity is then spoken of. In such a case, the weight gain is directly responsible for various health problems such as cardiovascular, articular or metabolic problems.

The factors responsible for weight gain are of two types: genetic factors on the one hand and lifestyle and alimentary behaviour on the other hand. The food and nutraceutical industries are currently paying attention to the second type of factor, taking an interest in the biological factors that participate in the physiological phenomena responsible for alimentary behaviour, and more particularly control of satiety. Disturbance of this control may not only be the cause of weight gain but also the cause of serious illnesses relating to disorders of the alimentary canal such as obesity, type II diabetes, cardiovascular problems, hypertension, atherosclerosis and hypercholesterolaemia.

Cholecystokinins, hereinafter referred to as CCKs, are a family of neuroendocrinal peptides. They are secreted at the small-intestine opening by enteroendocrinal cells, and at the central nervous system, which also confers on them a role in the transfer of information between the gastro-intestinal tract and the brain [1, 2]. The passage of the food through the duodenal part of the small intestine cause secretion of CCKs. This secretion cause numerous physiological processes such as intestinal mobility, contraction of the gall bladder, inhibition of gastric clearance, stimulation of pancreatic secretion and inducing the phenomenon of satiety [4]. The release of CCKs is due, in order of importance, to the action of protein, lipidic and glucidic compounds [6].

Previous works have shown the satietogenic potential exerted by certain protein hydrolysates in rats [7, 8], pigs [5] and humans [9].

The applicants also discovered that protein or peptide hydrolysates, obtained from the enzymatic hydrolysis of the muscle of certain fish had properties stimulating the secretion of CCKs by intestinal enteroendocrine cells.

GLP-1, glucagon-like peptide 1, is a gastro-intestinal hormone secreted by the epithelial cells of the intestine in response to the ingestion of nutriments.

GLP-1 regulates the metabolism of nutriments and elimination thereof by increasing the synthesis and secretion of insulin when glycaemia is too high (postprandial glycaemia). In parallel, GLP-1 restricts the release of glucagon, a hyperglycaemia-causing hormone, via the pancreatic islets.

GLP-1 also reducing digestive motricity and causes a sensation of satiety.

The invention also concerns a fish protein hydrolysate that is characterised in that it is obtained by enzymatic hydrolysis of at least one protein source chosen from the group composed of the pelagic fish species Micromesistius poutassou, Clupea harengus, Scomber scombrus, Sardina pilchardus, Trisopterus esmarki, Tracharus spp, the demersal fish species Gadus morhua, Pollachius virens, Melanogrammus aeglefinus, Coryphaenoides rupestris, and fish species belonging to the order Siluriformes, the said enzymatic hydrolysis being carried out by means of a mixture of enzymes comprising endopeptidases derived from Bacillus amyloliquefaciens and Bacillus licheniformis and in that it has:
the following molecular profile distribution: from 23% to 31% molecules with a molecular weight of less than 300 Da, from 31% to 34% molecules the molecular weight of which is between 300 and 1000 Da, from 28% to 34% molecules the molecular weight of which is between 1000 and 3000 Da, from 6% to 8% molecules the molecular weight of which is between 3000 and 5000 Da and 2% to 4% molecules the molecular weight of which is between 5000 and 10000 Da,
a lipid content of less than 1% as a percentage of raw product,
a glucid content of less than 0.1% as a percentage of raw product,
a protein content of more than 80% as a percentage of raw product,
a mineral matter content of between 10% and 20% as a percentage of raw product,

and in that it contains molecules immunologically similar to cholecystokinins, or CCKs.

The protein hydrolysate according to the invention contributes exogenous CCK molecules. It also stimulates the secretion of endogenous GLP1 molecules and CCK molecules by intestinal cells. The hydrolysate thus controls satiety, as demonstrated by the following examples.

According to one feature of the invention, the fish protein hydrolysate has the following amino acid composition: Glutamic acid 17.4%, Aspartic acid 11.4%, Lysine 10.2%, Leucine 8.4%, Arginine 6.1%, Alanine 6.8%, Valine 4.7%, Isoleucine 4.2%, Glycine 5%, Threonine 4.5%, Serine 4.4%, Tyrosine 3.2%, Phenylalanine 3.9%, Methionine 2.5%, Proline 3.6%, Histidine 1.9%, Cystine 1%, Tryptophan 0.8%, as a percentage by weight with respect to the total weight of amino acids.

According to a preferred embodiment of the invention, the said fish protein source is in the form of the pulp of the fillet of the said fish or fishes.

According to another embodiment of the invention, the said mixture of enzymes also comprises an endopeptidase derived form Aspergillus oryzae.

The present invention also concerns a method of obtaining a protein hydrolysate from a fish protein source having properties stimulating the secretion of CCKs and GLP1 at the level of the intestinal cells and capable of exerting a satietogenic effect as specified previously. The method according to the invention is characterised in that it comprises:
the grinding of at least one protein source chosen from the group composed of the fish species Micromesistius poutassou, Clupea harengus, Scomber scombrus, Sardina pilchardus, Trisopterus esmarki, Tracharus spp, the demersal fish species Gadus morhua, Pollachius virens, Melanogrammus aeglefinus, Coryphaenoides rupestris, and fish species belonging to the order Siluriformes in the presence of water, so as to recover the fish pulp,
the enzymatic hydrolysis of the said protein source at a temperature of between 40° and 63° C., at a pH situated between 6 and 9, for 1 to 5 hours, after the addition of a mixture of enzymes comprising endopeptidases derived from Bacillus amyloliquefaciens and Bacillus licheniformis, so as to obtain a reaction mixture,
stoppage of the said enzymatic hydrolysis by inactivation of the said enzymes after raising the temperature of the said reaction mixture to a level not below 70° C., for 8 to 20 minutes,
the separation of the protein hydrolysate obtained from the rest of the reaction mixture.

The enzymatic hydrolysis is carried out by means of a mixture of enzymes carefully selected so as to make it possible to obtain a protein hydrolysate having the aforementioned properties sought. The method, through the nature of the enzymes, the hydrolysis temperature and the absence of solvents, respects the organoleptic and nutritional qualities of the hydrolysates obtained. These hydrolysates can be incorporated in food products, neutraceutical compositions or pharmacalogical preparations.

According to an embodiment of the invention, the grinding of the protein source is carried out in the presence of water in accordance with a ratio by weight of protein source to water of 1.

According to an embodiment of the invention, the said enzymatic hydrolysis is carried out in accordance with a ratio of enzyme to protein source of between 0.01 and 2%. Preferentially, the ratio between enzyme and protein source is 0.5%.

According to an embodiment of the invention, the said enzymatic hydrolysis is carried out at a temperature of 60° C.

According to an embodiment of the invention, the said enzymatic hydrolysis is carried out at a pH of 7.5.

The separation of the protein hydrolysate obtained from the rest of the reaction mixture is generally carried by centrifugation at a speed of between 4000 and 7000 rev/min and elimination of the residue obtained. Preferentially, the separation of the protein hydrolysate obtained can be achieved by filtration of the said reaction mixture prior to the said centrifugation. The filtration of the reaction medium eliminates the solid matter.

According to an embodiment of the invention, the said method also comprises the concentration and atomisation or freeze drying of the said hydrolysate obtained.

According to an embodiment of the invention, the said enzymatic hydrolysis is stopped when the degree of hydrolysis reaches a maximum value of 9% and preferably between 8.75% and 8.95%.

According to an embodiment of the invention, the pH of the reaction mixture during hydrolysis is controlled and kept constant by the addition of sodium hydroxide at 1 mol.1⁻¹.

According to another embodiment of the invention, the said mixture of enzymes also includes an endopeptidase derived from Aspergillus oryzae.

The protein hydrolysate obtained after hydrolysis reaction in the presence of a mixture of three enzymes respectively derived from Bacillus amyloliquefaciens, Bacillus licheniformis and Aspergillus oryzae has the same properties and physical and chemical characteristics as a protein hydrolysate obtained after a hydrolysis reaction in the presence of a mixture of two enzymes derived respectively from Bacillus amyloliquefaciens and Bacillus licheniformis.

According to an advantageous embodiment of the method according to the invention, the mixture of enzymes is chosen from the CR 1020 mixture or the Protamex mixture. The CR 1020 mixture is sold by the company Meatzyme (Chr Winthersvej 36A, 2800 Kgs Lyngby, Denmark). The Protamex mixture is sold by the company Novozyme (Krogshoejvej 36, Denmark-2880 Bagsvaerd).

According to one embodiment of the invention, the said enzymatic hydrolysis is stopped by raising the temperature of the said reaction mixture to 90° C. and maintaining this temperature for 10 minutes.

According to a preferred embodiment of the invention, the said grinding of the said protein source is carried out from the fillet of the said fish or fishes.

The method according to the invention thus makes it possible to obtain a fish protein hydrolysate as described previously.

The present invention also concerns a composition, a food product and a food supplement comprising a fish protein hydrolysate as described previously.

The present invention also concerns a medication comprising a fish protein hydrolysate as described previously, and the use of such a fish protein hydrolysate for manufacturing a medication intended for the treatment of obesity and type II diabetes, and the prevention of cardiovascular problems, hypertension and atherosclerosis. This is because, as explained previously, the fish protein hydrolysate according to the invention can be used in the treatment or prevention of such pathologies. More particularly, the fish protein hydrolysate according to the invention can be used in the stimulation of the secretion of CCK molecules and/or in the stimulation of the secretion of GLP1 molecules.

The nutraceutical or pharmaceutical formulations incorporating a fish protein hydrolysate according to the invention can comprise ingredients normally used in this type of formulation such as binders, flavourings, preservatives or colourings and, in the case of food supplements or medications, may be in the form of tablets, granules or capsules. Formulations according to the invention can also be in the form of food products such as drinks, or in the form of suspensions or syrups.

The features of the invention mentioned above, as well as others, will emerge more clearly from a reading of the following description of an example embodiment, the said example being intended to be illustrative and non-limitative.

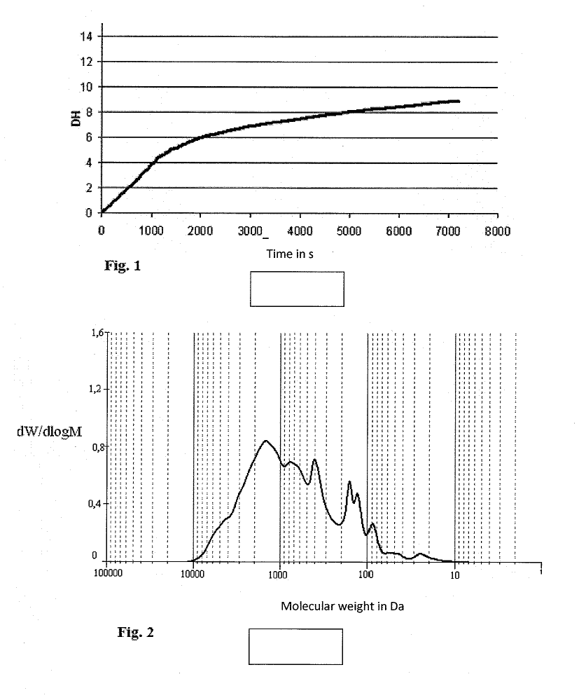

FIG. 1 illustrates the change in the degree of hydrolysis of the blue whiting protein hydrolysate according to the invention,

FIG. 2 illustrates the distribution of the molecular weights of the protein fragments of a blue whiting protein hydrolysate according to the invention, and

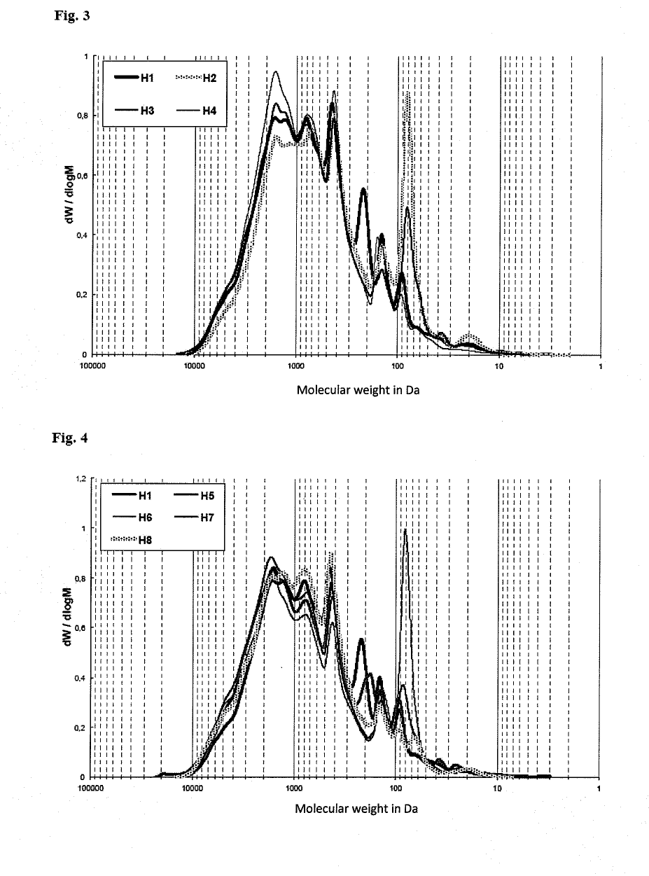

FIGS. 3 to 5 illustrate the distributions of the molecular weights of the protein fragments of protein hydrolysates of other species of fish according to the invention

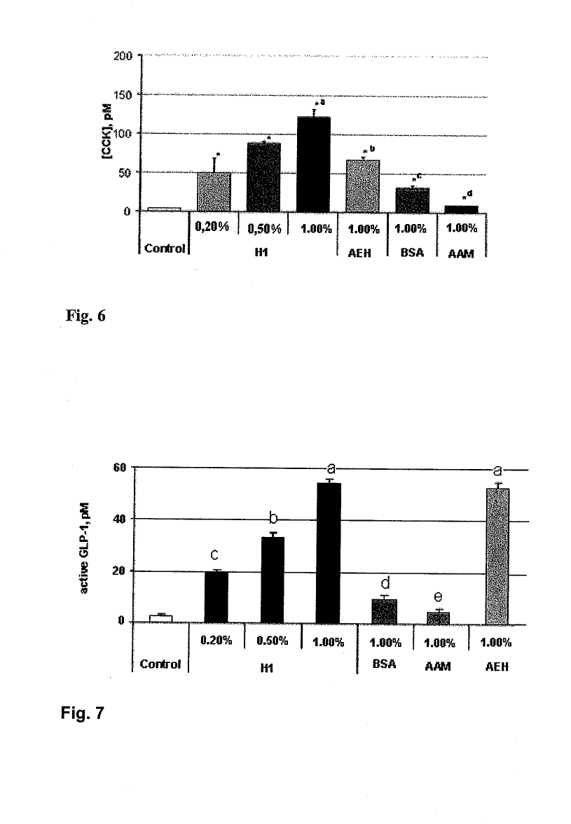

FIG. 6 illustrates the secretion of CCK molecules by the STC-1 cells in the presence or absence of a blue whiting protein hydrolysate according to the invention,

FIG. 7 illustrates the secretion of GLP1 molecules by the STC-1 cells in the presence or absence of a blue whiting protein hydrolysate according to the invention,

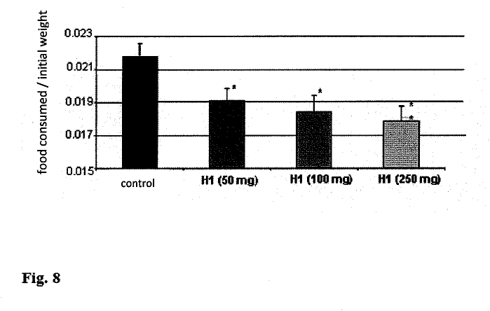

FIG. 8 illustrates an effect of a blue whiting protein hydrolysate according to the invention on food intake in rats, and

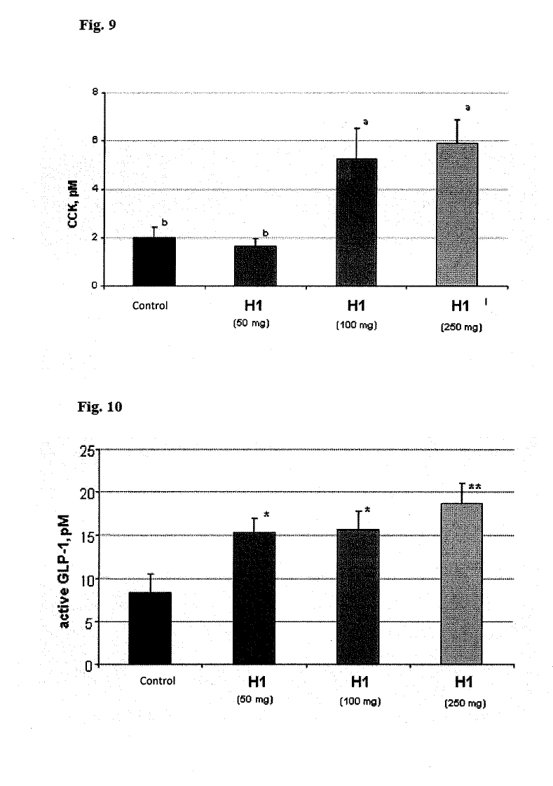

FIGS. 9 and 10 show the plasmatic dosages of CCK and GLP1 molecules respectively in rats after the absorption or not of a blue whiting protein hydrolysate according to the invention.

### EXAMPLE 1

### PROTEIN HYDROLYSATE OBTAINED FROM BLUE WHITING, MICROMESISTIUS POUTASSOU (H1)

Blue whiting (Micromesistius poutassou) is fished in the North Atlantic off Newfoundland. The fish are cut into fillets, which are then ground so as to obtain the pulp. This fish pulp constitutes a source of protein for the production of hydrolysate. The pulp is stored at −20° C. until used.

Three kilograms of blue whiting pulp previously thawed are mixed with water in a ratio by weight of 1. The temperature of the mixture is raised to 60° C. and the pH is adjusted to 7.5 by means of a sodium hydroxide 1M solution, under agitation.

A mixture composed of three enzymes, respectively derived from Bacillus amyloliquefaciens, Bacillus licheniformis and Aspergillus oryzae, and sold under the name CR 1020 by the company Meatzyme (Chr Winthersvej 36A, 280 Kgs Lyngby, Denmark) is then added to the reaction mixture in an enzyme/protein source ratio of 0.5%. During the hydrolysis reaction, the pH is kept constant at 7.5 by the addition of sodium hydroxide 1M (NaOH).

The blue whiting protein hydrolysis reaction is carried out for 2 hours under controlled conditions by means of the well known so-called pH-STAT method. The pH-STAT method is based on keeping the pH constant during the hydrolysis reaction. The extent of the hydrolysis is thus quantified by the degree of hydrolysis (DH), which is determined by the number of peptide bonds cut over the total number of peptide bonds.

The DH is calculated from the volume and molarity of the base used for keeping the pH constant. As long as the pH remains constant, there is a relationship between the number of hydrolysed bonds and the volume of sodium hydroxide poured. For a given enzymatic system and a constant pH, the functionality will be the same from one hydrolysate to another, if the reaction is stopped each time at the same DH.

% DH=[(B.NB)/(a.htot.MP)]*100

with:
B the consumption of the base (in ml or l)
NB the normality of the base
α the mean dissociation of the HN or COOH groups
MP the mass of proteins (determined by the Kjeldhal method, in g or kg)
htot the total number of peptide bonds

After 2 hours of hydrolysis reaction, the final DH of the protein hydrolysate is 8.9% (FIG. 1).

The inactivation of the enzymes at the end of the hydrolysis kinetics is achieved by increasing the temperature of the reaction medium up to 90° C. This temperature is maintained for 10 minutes.

The blue whiting protein hydrolysate obtained, hereinafter referred to as H1, is then filtered on a sieve (mesh 2 mm/2 mm) so as to eliminate the solid matter. The fraction recovered in the receptacle is then centrifuged for 30 minutes ±5 minutes, at a speed of between 4000 and 7000 rev/min. After elimination of the remainder, the supernatant is recovered, freeze dried and stored in a cool dry place, away from light. The supernatant may also be atomised.

In a variant of the invention, it is possible to deactivate the endogenous enzymes by increasing the temperature to boiling point prior to the addition of the mixture of aforementioned enzymes.

In another variant of the invention, the enzymatic hydrolysis is performed using a mixture composed of two enzymes respectively derived from Bacillus amyloliquefaciens and Bacillus licheniformis and sold under the name Protamex by the company Novozyme (Krogshoejvej 36, Denmark-2880 Bagsvaerd).

Physical and Chemical Analyses of the Protein Hydrolysate Obtained from Blue Whiting

A determination of the molecular weights of the peptides making up the protein hydrolysate obtained is carried out by steric exclusion chromatography (SEC-HPLC).

The protein hydrolysate H1, in the form of powder after freeze drying, is suspended in ultra-pure water (20 mg/ml) and then filtered on a 0.45 μm membrane and analysed by filtration over gel with a Superdex Peptide HR 10/30 column, sold by the company Pharmacia. The matrix of the column is composed of a crosslinked porous gel (diameter 13-15 μm) of agarose and dextran with a total volume of 24 ml. Its fractionation domain is between 100 and 7000 Da. The column is mounted on an HPLC line (sold by the company Dionex) equipped with a pump (Dionex P680 module). The measurement is carried out by a multi-wavelength ultraviolet detector (Dionex UVD 170 U module). The protein hydrolysate is eluted by a mobile phase containing acetonitrile, water and TFA. The elution lasts for approximately 1 hour at a rate of 0.5 ml/min.

The distribution of molecular weights is calculated from the parameters of a calibration line obtained after passage through the column of markers with known molecular weights. These markers are Cytochrome C (12,400 Da), aprotinin (6511 Da), gastrin I (2126 Da), the substance P (1348 Da), the substance P fragment 1-7 (900 Da), glycine (75 Da) and leupeptin (463 Da). The data are collected by means of Chromeleon software (Dionex). The percentages of the molecular weights are calculated by means of software (GPC Cirrus from Polymer Laboratories). The acquisition wavelength is 214 nm. The distribution of the molecular weights as a function of dW/log M is given on FIG. 2, and the distribution of the molecular weights by class of size is given in table 2 below. The percentage of the area under the curve corresponds to the percentage of peptide molecules.

The amino acid composition of the blue whiting protein hydrolysate H1 is given in table 1 (according to European directive 98/64/CE and NF EN ISO 13904-October 2005). Table 2 shows the distribution of the amino acids in the H1 protein hydrolysate.

TABLE 1

| | Percentage of |
|---|---|
| Amino acid | amino acid |
| Glutamic acid | 17.4 |
| Lysine | 10.2 |
| Aspartic acid | 11.4 |
| Leucine | 8.4 |
| Arginine | 6.1 |
| Alanine | 6.8 |
| Valine | 4.7 |
| Isoleucine | 4.2 |
| Cystine | 1 |
| Glycine | 5 |
| Threonine | 4.5 |
| Serine | 4.4 |
| Tyrosine | 3.2 |
| Phenylalanine | 3.9 |
| Methionine | 2.5 |
| Proline | 3.6 |
| Histidine | 1.9 |
| Tryptophan | 0.8 |

The protein content is above 80%, as a percentage of raw product (according to NF V18-120-March 1997-corrected KJELDAHL).

The lipid content is less than 1%, as a percentage of raw product (according to European Directive 98/64/CE).

The energy value of the protein hydrolysate H1 is approximately 330 Kcal/100 g.

The glucid content is less than 0.1% (deduced from the protein and glucid contents and the energy value).

### EXAMPLE 2

### PROTEIN HYDROLYSATE OBTAINED FROM OTHER SPECIES OF FISH ACCORDING TO THE INVENTION

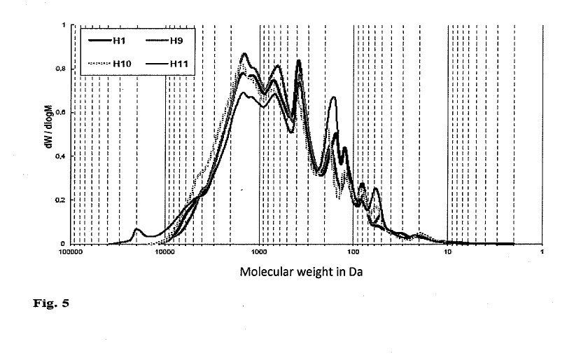

Hydrolysates of proteins of mackerel (H2) (Scomber scombrus), horse mackerel (H3) (Trachurus spp.), grenadier (H4) (Coryphaenoides rupestris) (FIG. 3); bib (H5) (Trisopterus esmarki), sardine (H6) (Sardina pilchardus) herring (H7) (Clupea harengus), panga (H8) (Suliform) (FIG. 4); cod (H9) (Gadus morhau), pollock (H10) (Pollachius virens) and haddock (H11) (Melanogrammus aeglefinus) (FIG. 5) were prepared according to the method of example 1. The distribution of the molecular weights of the peptides making up each hydrolysate was analysed according to the same method as that used in example 1.

The distribution of the molecular weights as a function dW/log M is given in FIGS. 3 to 5, and the distribution of the molecular weights by class of size is given in the following table 2. The percentage of the area under the curve corresponds to the percentage of peptide molecules.

TABLE 2

| Classes | H1 | H2 | H3 | H4 | H5 | H6 | H7 | H8 | H10 | H9 | H11 |
|---|---|---|---|---|---|---|---|---|---|---|---|
| <0.3 | 23-31 | 30 | 23 | 23 | 23 | 25 | 23 | 23 | 24 | 24 | 29 |
| 0.3-1 | 31-34 | 33 | 34 | 33 | 32 | 31 | 32 | 33 | 31 | 34 | 33 |
| 1-3 | 28-34 | 29 | 33 | 34 | 33 | 32 | 33 | 34 | 33 | 34 | 28 |
| 3-5 | 6-8 | 6 | 7 | 7 | 8 | 8 | 8 | 7 | 8 | 6 | 6 |
| 5->10 | 2-4 | 2 | 3 | 3 | 4 | 4 | 4 | 3 | 4 | 2 | 4 |

Hydrolysates H1 to H11 have identical molecular weight distribution profiles.

### EXAMPLE 3

### ANALYSIS OF MOLECULES IMMUNOLOGICALLY SIMILAR TO CHOLECYSTOKININS (CCKS) IN THE BLUE WHITING PROTEIN HYDROLYSATE H1

The molecules similar to CCKs present in the protein hydrolysate H1 were analysed by radioimmunological analysis using the RIA kit (GASK-PR, CIS Bio International, Bagnols/Cèze, France) (test carried out in triplicate). Molecules similar to CCKs means any molecules capable of being fixed to a specific antibody directed against the eight amino acids common to gastrins and CCK, the said antibody being the antibody used in the aforementioned analysis. Gastrin and CCKs have identical peptide sequences in the C-terminal part of their peptide sequences.

The protein hydrolysate contains 5.6 pg of molecules similar to gastrins/CCKs per mg of dry weight of hydrolysate.

The hydrolysate according to the invention thus makes possible a supply of molecules similar to CCK molecules.

### EXAMPLE 4

### IN VITRO CELL CELTURE TEST: EFFECT ON THE STIMULATION OF THE SECRETION OF CCK MOLECULES AND ON THE STIMULATION OF THE SECRETION OF GLP1 MOLECULES

The H1 protein hydrolysate was tested for its ability to stimulate the secretion of CCK molecules, as well as GLP1, at type STC1 enteroendocrine cells. This is because the secretion of CCK and GLP1 by intestinal endocrine cells represents one of the main signals constituting the phenomenon of satiety. STC-1 cells are plurihormonal cells derived from tumoral endocrine cells issuing from the small intestine of a mouse. STC-1 cells are used as a cell model for the study of phenomena giving rise to the specific secretion of CCK [10] and as a cell model for the study of phenomena causing the specific secretion of GLP1 [11].

STC-1 cells were cultivated in DMEM medium containing 2 mM of 1-glutamine, 2 mM of penicillin, 50 μm of streptomycin and 10% of foetal calf serum (FCS). Between 2 and 3 days before the test was carried out, the STC-1 cells were put in cultures in 24-well plates at the rate of 30,000 to 40,000 cells per well. When the cells reached a confluence level of approximately 85%, the wells were rinsed twice with incubation buffer (4.5 mM KCl, 1.2 mM CaCl₂, 1.2 mM MgCl₂, 10 mM glucose, 140 mN NaCl and 20 mM Hepes-Tris, pH 7.4).

The cells were then incubated for 2 hours in the presence of various solutions composed either of bovine serum albumin (BSA), or H1 protein hydrolysate at different concentrations, or free amino acids (cf. table 3), or a commercial albumin egg hydrolysate (AEH) or incubation buffer used as a culture reference (control). The supernatants of the cultures were centrifuge (5 minutes, 2000 g). After centrifugation, the supernatants were recovered and stored at -20° C. before analysing the CCK content by radioimmunological analysis using the RIA kit (GASK-PR, CIS Bio International, Bagnols/Cèze, France) (STC-1 cells do not secret gastrins). The analysis is carried out according to the protocol supplied by the distributor. The GLP-1 concentrations in its active form secreted by the STC-1 cells were determined using the radioimmunological analysis kit (GLP1A-35 HK, Linco Research, St Charles, Mo. USA). The analysis is carried out in accordance with the protocol provided by the distributor.

The results concerning the analysis of the CCK molecules are presented in FIG. 6 and are expressed in picomoles/1 (pM) of CCK excreted by the cells. The results concerning the analysis of the GLP1 molecules are presented in FIG. 7 and are expressed in picomoles/1 (pM) of GLP1 excreted by the cells.

In FIGS. 6 and 7, the sign * designates the values significantly different from the value obtained for the control (t-test (P<0.05)). The letters (a, b, c, d) represent values significantly different from one another (t-test, P<0.05).

TABLE 3
Composition of the free amino acid solution

| | Quantity |
|---|---|
| Amino acid | (mg/l) |
| L-glutamic Acid | 151 |
| L-Lysine | 88 |
| L-aspartic acid | 151 |
| L-Leucine | 112 |
| L-Arginine | 277 |
| L-Alanine | 96 |
| L-Valine | 80 |
| L-Isoleucine | 64 |
| L-Cysteine | 16 |
| Glycine | 128 |
| L-Threonine | 64 |
| L-Serine | 26 |
| L-Tyrosine | 06 |
| L-Phenylalanine | 91 |
| L-Methionine | 40 |
| L-Proline | 53 |
| L-Histidine | 72 |
| L-Tryptophan | 16 |

The results show the significant effect of the H1 blue whiting protein hydrolysate at different concentrations (0.2; 0.5 and 0.1% mass/volume ratio), on the secretion both of CCK molecules and GLP1 molecules in the extracellular medium by the STC-1 cells compared with the other solutions tested.

The quantities of CCK and CLP1 molecules released by the cells increase significantly with the concentrations of hydrolysate (FIGS. 6 and 7). The quantity of CCK molecules obtained from 1.0% concentrated hydrolysate was 30 times greater than that obtained with the reference culture (122.03 pM of CCK as against 4.02 pM of CCK respectively). The quantities of CCK obtained in the presence of BSA or free amino acids at 1.0% are considerably less than those obtained in the presence of hydrolysates with the same concentration (31.2 and 8.6 pM respectively). The same observations are found with regard to the secretion of GLP1 molecules.

These results indicate that the blue whiting protein hydrolysate contains molecules capable of greatly stimulating the secretion of CCK and GLP1 molecules by the STC-1 cells. The low stimulating potentials of solutions of BSA and free amino acid solutions indicate that the stimulating effect of the secretion of CCK and GLP1 molecules is not due either to a “protein effect” or to the action of free amino acids present in large numbers in the blue whiting hydrolysate. This stimulation therefore appears to be mainly due to the peptide molecules contained in the H1 protein hydrolysate.

### EXAMPLE 5

### DEMONSTRATION OF THE SATIETOGENIC EFFECT OF A PROTEIN HYDROLYSATE ACCORDING TO THE INVENTION IN RATS—IN VIVO STUDY

The properties of the H1 hydrolysate obtained according to example 1 were evaluated on the food intake in rats, as well as on various blood parameters. The purpose being to demonstrate a satietogenic effect of the hydrolysate on food intake, corroborated by endocrinal physiological parameters.

Experimental Protocol:

32 male Wistar rats weighing 275 to 290 grams (Harlan, France) divided into 4 groups of 8 individuals are kept in opaque individual cages closed by an aluminium grille (to deprive them of the sight of other rats and not to interfere with their feeding). They are installed in a room air-conditioned at a temperature of 21±1° C. and a day:night cycle of 12 hours (5 pm-7 am). They have ad libitum access to food (Complete Food, Harlan, France) and water during the adaptation period (5 days) and the first two weeks of experimentation.

Each group of rats is force fed with different force feeding compositions:

group 1: water

group 2: H1 (50 mg.day⁻¹)

group 3: H1 (100 mg.day⁻¹)

group 4: H1 (250 mg. day⁻¹)

Each rat is force fed individually in a separate room, out of view of the other rats, with 0.5 ml of water or dissolved H1 hydrolysate (50, 100 or 250 mg/mL⁻¹ according to the group). The duration of the force feeding can be estimated at three minutes per rat and makes it possible to measure the food intake (by weighing the food consumed) in parallel, the complete food is presented to the rat 10 minutes after the force feeding. The litters are also changed at the time of force feeding once per week and the rats are weighed once a week on Mondays.

1. Food Intake

After having been weighed in order to form groups with equivalent mean weights, the rats are kept in an adaptation period for the seven days preceding the start of the experimentation. The experimentation phase begins on the Monday (D1) of the first week and continues for two weeks.

On the first day of the experimentation (D1) in the morning, the rats are made to fast for 24 hours. On the second day (D2) in the morning, the rats are weighed and undergo blood sampling at the end of the tail (collection in tubes containing 5% EDTA), following which the complete food is made available to them again. The blood samples are then centrifuged and the plasma is stored at −20° C.

The rats in the 4 groups are force fed on a first occasion with their respective force-feeding composition orogastrically by means of an intragastric probe on the second day (D2) at 5 pm. A first measurement of the food intake by weighing takes place 2 hours later. On the morning of the third day (D3), the food intake is once again assessed before force feeding carried out at 9 am and then a measurement of the food intake is once again carried 3 hours later. The rats are once again force fed at 5 pm and at the same time the food intake is measured, and then once again measured 2 hours later. These steps of force feeding and measurements are carried out until the fifth day (D5) in the evening inclusive, and repeated for a second week from Monday (day D8) in the morning to Friday (day D12) in the evening.

2. Plasmatic Hormones

At the end of the second week, the rats are sacrificed by decapitation 30 minutes after force feeding and the blood is sampled on tube/5% EDTA. 3 aliquots of plasma will then be produced in order to analyse the following circulating hormones:

aliquot 1: GLP-1

aliquot 2: CCK

Results

1. Food Intake

The results presented in FIG. 8 express the added food consumption of the rats, in grams of food and according to their initial weight, over the whole of the two experimentation weeks. The values are the means of the daily values obtained for each group during the experimentation, and expressed ±SEM; *: p<0.05, **p<0.01.

Thus the results show that, between 9 am and 7 pm, the values relating to the food intake obtained for the groups 2, 3 and 4 that received H1 are significantly less than those obtained for the reference group 1, which did not receive H1. A reduction in the food intake according to the daily dose of H1 hydrolysate is observed; although there is no significant difference between groups 2, 3 and 4, the probability that there exists a difference between the reference group 1 and the other groups 2, 3, 4 increases with the daily dose of hydrolysate administered.

2. Plasmatic Hormones

The concentrations of CCK in the plasmas of rats were measured by means of a radio-immunological analysis developed by the Compagnie de Pêches Saint Malo-Sante. This analysis has the particularity of using a specific antibody, developed in 1998 (Rehfeld 1998), for the sulphated active CCKs, which do not cross with the various forms of gastrin (present in the plasma in larger quantities than the CCK). This protocol was developed in particular from an analysis kit distributed by IBL (IBL, Hamburg, Germany) using the same antibody.

The concentrations of GLP-1 in its plasmatic active form were determined by means of the radio-immunological analysis kit (GLP1A-35HK, Linco Research, St Charles, Mo., USA). The analysis is carried out in accordance with the protocol supplied by the distributor. The results are presented in FIGS. 9 and 10. The plasmatic concentration of GLP-1 (FIG. 10) and CCK (FIG. 9) are expressed in pmol.l-1 of blood plasma. The values are the means of each group, expressed ±SEM. *: T-test, p<0.05, **: T-test, p<0.01. In FIG. 9, the averages not assigned an identical letter are different.

It should be noted that the plasmatic GLP1 concentrations are significantly different from that obtained with the reference, whatever the dose of hydrolysate received by the animal. The plasmatic CCK concentrations for the groups that received 100 or 250 mg.day⁻¹ of H1 are significantly different from that obtained with the reference. This analysis joins the analysis of the food intake.

### BIBLIOGRAPHY

[1] Strader A D, Woods S C. Gastrointestinal hormones and food intake. Gastroenterology 2005; 128:175-191
[2] Moran T H, Kinzig K P. Gastrointestinal satiety signals II. Cholecystokinin. Am. J. Physiol. Gastrointest. Liver Physiol. 2004; 286:G183-188.
[3] Moran T H. Cholecystokinin and satiety: current perspectives. Nutrition 2000; 16:858-865
[4] Chaudhri O, Small C, Bloom S. Gastrointestinal hormones regulating appetite. Philos Trans R Soc Lond B Biol Sci 2006; 361:1187-209.
[5] Cuber J C, Bernard C, Levenez F, Chayvialle J A. [Lipids, proteins and carbohydrates stimulate the secretion of intestinal cholecystokinin in the pig]. Reprod Nutr Dev 1990; 30:267-75.
[6] Baile C A, McLaughlin C L, Della-Fera M A. Role of cholecystokinin and opioid peptides in control of food intake. Physiol Rev 1986; 66:172-234.
[7] Liddle R A, Green G M, Conrad C K, Williams J A. Proteins but not amino acids, carbohydrates, or fats stimulate cholecystokinin secretion in the rat. Am J Physiol 1986; 251:G243-8.
[8] Douglas B R, Woutersen R A, Jansen J B et al. The influence of different nutrients on plasma cholecystokinin levels in the rat. Experientia 1988; 44:21-3.
[9] Miazza B, Palma R, Lachance J R et al. Jejunal secretory effect of intraduodenal food in humans. A comparison of mixed nutrients, proteins, lipids, and carbohydrates. Gastroenterology 1985; 88:1215-22.
[10] Mangel A W et al. Phenylalanine-stimulated secretion of cholecystokinin is calcium dependent. Am J Physiol, 1995. 268(1 Pt 1): p. G90-4.
[11] Brubaker P L, Izzo A, Rocca A S. Synthesis and secretion of intestinal proglucagon-derived peptides by the STC-1 enteroendocrine cell line. Can J Diabetes. 2003;27:141-148.

1. Fish protein hydrolysate characterised in that it is obtained by enzymatic hydrolysis of at least one protein source chosen from the group composed of the fish species Micromesistius poutassou, Clupea harengus, Scomber scombrus, Sardina pilchardus, Gadus morhua, Pollachius vixens, Melanogrammus aeglefinus, Coryphaenoides rupestris, Trisopterus esmarki, Tracharus spp, and fish species belonging to the order Siluriformes, the said enzymatic hydrolysis being carried out by means of a mixture of enzymes comprising endopeptidases derived from Bacillus amyloliquefaciens and Bacillus licheniformis and in that it has:
the following molecular profile distribution: from 23% to 31% molecules with a molecular weight of less than 300 Da, from 31% to 34% molecules the molecular weight of which is between 300 and 1000 Da, from 28% to 34% molecules the molecular weight of which is between 1000 and 3000 Da, from 6% to 8% molecules the molecular weight of which is between 3000 and 5000 Da and 2% to 4% molecules the molecular weight of which is between 5000 and 10000 Da,
a lipid content of less than 1% as a percentage of the raw product,
a glucid content of less than 0.1% as a percentage of raw product,
a protein content of more than 80% as a percentage of the raw product,
a mineral matter content of between 10% and 20% as a percentage of raw product, and in that it contains molecules immunologically similar to cholecystokinins (CCKs).

2. Fish protein hydrolysate according to claim 1, characterised in that it has the following amino acid composition: Glutamic acid 17.4%, Aspartic acid 11.4%, Lysine 10.2%, Leucine 8.4%, Arginine 6.1%, Alanine 6.8%, Valine 4.7%, Isoleucine 4.2%, Glycine 5%, Threonine 4.5%, Serine 4.4%, Tyrosine 3.2%, Phenylalanine 3.9%, Methionine 2.5%, Proline 3.6%, Histidine 1.9%, Cystine 1%, Tryptophan 0.8%, as a percentage by weight with respect to the total weight of amino acids.

3. Fish protein hydrolysate according to claim 1, characterised in that the said source of fish proteins comprises the pulp obtained from the fillet of the said fish or fishes.

4. Fish protein hydrolysate according to claim 1, characterised in that the said mixture of enzymes also comprises an endopeptidase derived from Aspergillus oryzae.

5. Method of obtaining a fish protein hydrolysate as defined in claim 1, characterised in that it comprises:
the grinding of at least one protein source chosen from the group composed of the fish species Micromesistius poutassou, Clupea harengus, Scomber scombrus, Sardina pilchardus, Gadus morhua, Pollachius virens, Melanogrammus aeglefinus, Coryphaenoides rupestris, Trisopterus esmarki, Tracharus spp, and fish species belonging to the order Siluriformes, in the presence of water, so as to recover the pulp from the said fish or fishes,
the enzymatic hydrolysis of the said protein source at a temperature of between 40° and 63° C., at a pH situated between 6 and 9, for 1 to 5 hours, after the addition of a mixture of enzymes comprising endopeptidases derived from Bacillus amyloliquefaciens and Bacillus licheniformis, in a ratio of enzyme to protein source of between 0.01 and 2%, so as to obtain a reaction mixture,
stoppage of the said enzymatic hydrolysis by inactivation of the said enzymes after raising the temperature of the said reaction mixture to a level not below 70° C., for 8 to 20 minutes,
the separation of the protein hydrolysate obtained from the rest of the reaction mixture.

6. Method according to claim 5, characterised in that the said enzyme/protein source ratio is 0.5%, the said hydrolysis temperature is 60° C. and the said pH is 7.5.

7. Method according to claim 5, characterised in that the said stoppage of the said enzymatic hydrolysis is done when the degree of hydrolysis reaches 8.9.

8. Method according to claim 5, characterised in that the said mixture of enzymes also contains an endopeptidase derived from Aspergillus oryzae.

9. Composition characterised in that it comprises a fish protein hydrolysate as defined in claim 1.

10. (canceled)

11. (canceled)

12. Pharmaceutical composition characterised in that it comprises a fish protein hydrolysate as defined in claim 1.

13. (canceled)

14. Use of a fish protein hydrolysate as defined in claim 1 for manufacturing a medication intended for the treatment of obesity, type II diabetes, hypertension, atherosclerosis or hypercholesterolemia or the prevention of cardiovascular problems.

15. Fish protein hydrolysate as defined in claim 1 for use thereof in the treatment of obesity, type II diabetes, hypertension, atherosclerosis or hypercholesterolemia or the prevention of cardiovascular problems.

16. Fish protein hydrolysate as defined in claim 1 for use thereof in the stimulation of the secretion of CCK molecules and/or GLP 1 molecules.

17. Fish protein hydrolysate as defined in claim 1 for use thereof in the control of satiety.

18. Composition according to claim 9, characterised in that it is in the form of a food product, a food supplement or a neutraceutical composition.
